(19) United States
(12) Patent Application Publication
(10) Pub. No.: US 2022/0206017 A1
(43) Pub. Date: Jun. 30, 2022

(54) AEROBIC GLYCOLYSIS AND HYPERMETABOLIC STATES

(71) Applicant: Joel Steven Goldberg, Hillsborough, NC (US)

(72) Inventor: Joel Steven Goldberg, Hillsborough, NC (US)

(21) Appl. No.: 17/698,053

(22) Filed: Mar. 18, 2022

(51) Int. Cl. G01N 33/72; G01N 33/50

(52) U.S. Cl. CPC G01N 33/721; G01N 2800/22; G01N 33/5008

(57) ABSTRACT

Hypermetabolic states that include cancer, infections, inflammations, mental illnesses, and chronic pain are sustained with ATP produced by aerobic glycolysis. Because of redundancies within this metabolic pathway, inhibition of aerobic glycolysis requires a combination drug therapy, which at the present time is optimal with 2-Deoxy-D-Glucose (2DG) and D-Lactic Acid Dimer (DLAD). Red blood cells exclusively derive energy from aerobic glycolysis, and spectrophotometric measurement of hemolysis can be utilized to monitor the effectiveness of drug combinations that inhibit aerobic glycolysis, particularly in hypermetabolic states. These measurements can be performed cheaply and easily in most health care facilities. Complete inhibition of aerobic glycolysis may necessitate red blood cell transfusion, but cells with mitochondria can produce sufficient ATP through lipid oxidation and amino acid metabolism to remain viable.

### CROSS-REFERENCES TO RELATED APPLICATIONS

None

### FEDERALLY FUNDED RESEARCH

Not applicable

### BACKGROUND OF THE INVENTION

Hypermetabolic states are associated with a number of diseases including cancer, inflammations, infections, mental disorders, and chronic pain. These hypermetabolic states utilize aerobic glycolysis for the majority of adenosine triphosphate (ATP) production. This observation was first recognized in cancer cells by Otto Warburg in the 1920s. This invention extends prior work on the inhibition of aerobic glycolysis and monitoring of this inhibition through spectrophotometric measurement of free hemoglobin from red blood cell lysis.

### DESCRIPTION OF THE DRAWINGS

FIG. 1 is a cell diagram. Label 1 depicts the location of ATP production within the inner mitochondrial matrix. Label 2 depicts the location of ATP in the cytoplasm.

FIG. 2 is a diagram of glycolysis. Label 1 depicts inhibition of glyceraldehyde 3-phosphate dehydrogenase by 3-Bromopyruvate (3BP). Label 2 depicts inhibition of hexokinase II by 2-Dexoxy-D-Glucose (2DG). Label 3 depicts the effects of D-Lactic Acid Dimer (DLAD) on hydrogen ion transport and pyruvate concentration.

FIG. 3 shows the cytotoxicity of DLAD compared to LLAD and PBS.

### DETAILED DESCRIPTION OF THE INVENTION

Contrary to mainstream beliefs, aerobic glycolysis may be the most efficient pathway for the production of ATP to fulfil the energy needs of hypermetabolic states. L-Lactic acid, which is the end product of aerobic glycolysis, is only known to be produced by glycolysis from the reduction of pyruvate by lactate dehydrogenase (LDH), and more preferentially by the isoenzyme LDH1. At pH 7.4, L-Lactic acid is dissociated to L-Lactate at a ratio of approximately 1:4,000, respectively. Therefore, L-Lactate concentration is a measure of aerobic glycolysis.

Location of ATP Production

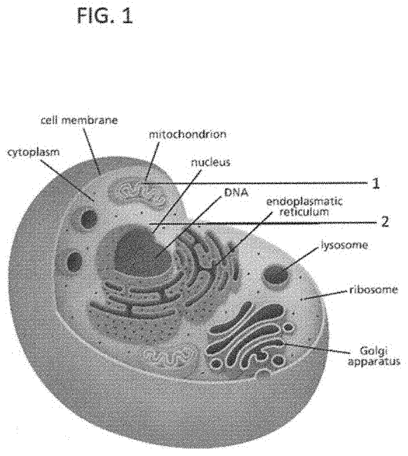

Without having to transverse two membranes (inner and outer mitochondrial) (FIG. 1, Label 1), aerobic glycolysis, which occurs in the cytoplasm (FIG. 1, Label 2), produces ATP that can be used in other organelles such as the nucleus, endoplasmic reticulum, ribosome, peroxisome, lysosome, and Golgi apparatus. Therefore, ATP produced by aerobic glycolysis is more available to organelles than ATP produced in the mitochondria.

Rate of ATP Production

Pyruvate must be transported from the cytoplasm to the mitochondrial inner matrix for GTP and ATP to be synthesized in the citric acid cycle and by oxidative phosphorylation. The time needed for this transport has not been determined, and it clearly affects the rate of ATP synthesis. At the present time, there are no methods that directly and simultaneously measure ATP production during glycolysis and the citric acid cycle and oxidative phosphorylation. All present measurements are indirect. [1] Therefore, it cannot be precisely determined that aerobic glycolysis is a less efficient source of ATP compared to synthesis in the citric acid cycle and oxidative phosphorylation. On a molar basis of glucose consumed, oxidative phosphorylation is more efficient than aerobic glycolysis. However, rate of ATP production and efficiency of glucose oxidation are not equivalent measurements.

Inhibitors of Aerobic Glycolysis

The ATP produced in the cytoplasm by aerobic glycolysis can be inhibited by drugs such as 3-Bromopyruvate (3BP), Dichloroacetate (DCA), 2-Deoxy-D-Glucose (2DG), and D-Lactic Acid Dimer (DLAD).

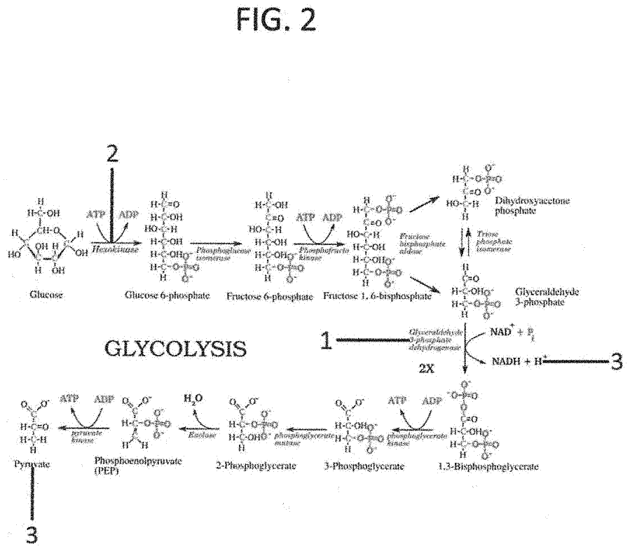

The drug 3BP primarily blocks glyceraldehyde 3-phosphate dehydrogenase activity which inhibits aerobic glycolysis, but does not block the upstream pentose phosphate pathway. (FIG. 2, Label 1) It has been shown that 3BP has significant alkylation properties which could affect DNA. Previous descriptions that it significantly inhibited hexokinase II were incorrect.[2] Its intravenous administration is often associated with pain and phlebitis.

DCA blocks the activity of pyruvate dehydrogenase kinase, which is a mitochondrial enzyme that inhibits pyruvate dehydrogenase the enzyme needed to convert pyruvate to Acetyl-CoA. [3] It shifts metabolism away from aerobic glycolysis toward oxidative phosphorylation. It does not selectively block aerobic glycolysis in hypermetabolic states since its actions are located in the mitochondria, and its clinical use is associated with neurotoxicity. It is administered intravenously.

The inhibition of aerobic glycolysis from 2DG is mainly from the inhibition of hexokinase II and not only blocks glycolysis but also the pentose phosphate pathway, which produces five carbon sugars needed for cell proliferation and NADPH. (FIG. 2, Label 2) However, other sugars such as fructose, can enter glycolysis downstream without phosphorylation by hexokinase II. The Ki of 2DG is report as 2.9 mM. Thus, complete block of glycolysis with only 2DG is not possible, and as a monotherapy 2DG will not be clinically effective. The 2DG can be administered orally and its side effects are generally well tolerated.

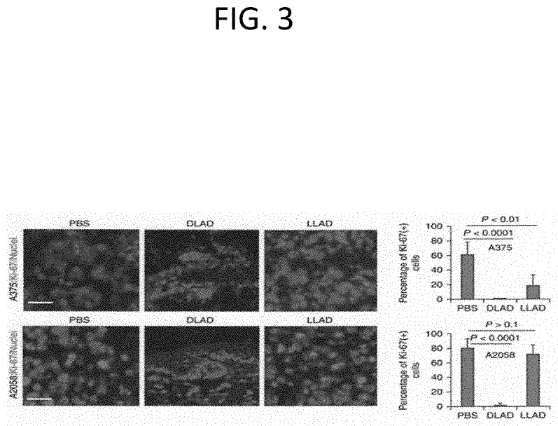

DLAD blocks glycolysis through spontaneous, non-enzymatic sequestration of L-lactate. This is a unique mechanism of drug action related to chiral properties of L-lactate and DLAD, and the mechanism of action is more closely related to drug chelation than enzyme inhibition. Sequestration of L-lactate interferes with the cell's ability to buffer the hydrogen ions produced in aerobic glycolysis as a consequence of glyceraldehyde 3-phosphate conversion to 1,3-biphosphoglycerate. (FIG. 2, Label 3) This not only leads to intracellular acidosis, but increases extracellular pH which limits the spread of cancer. Furthermore, when L-lactate is sequestered, this decreases the pyruvate available in the cytosol that can be transported to the mitochondria, since the sequestration of L-lactate produces a sink for the conversion of pyruvate to L-lactate. (FIG. 2, Label 3) In vivo studies show impressive melanoma cytotoxicity from DLAD as measured by Ki-67 nuclei compared to L-Lactic Acid Dimer (LLAD) and phosphate buffered saline control. (FIG. 3) [4] It is not known if DLAD can be orally adsorbed or the potential bioavailability of an ester prodrug. The combination of 2DG and DLAD is predicated to inhibit aerobic glycolysis significantly, which is the main metabolic pathway for production of ATP in hypermetabolic states.

Cells that do not need large quantities of ATP compared to those that are hypermetabolic are expected to be spared from inhibition of aerobic glycolysis, since they can produce Acetyl-CoA in the mitochondria through lipid oxidation or from metabolism of amino acids.

Waste Management of Aerobic Glycolysis

The fate of L-lactate produced in aerobic glycolysis is twofold. The L-lactate can be transported out of the cell and converted to pyruvate predominately in the liver by LDH1 via the Cori cycle. Also, the excess L-lactate, which is extracellular can be converted to bicarbonate and excreted in the lungs as carbon dioxide. Using these processes, the waste products of aerobic glycolysis can be efficiently managed, which implies that the metabolic pathway is an efficient system.

Red Blood Cell Viability as a Model of Aerobic Glycolysis Activity

Red blood cells (RBC) do not contain mitochondria and exclusively rely on glycolysis for production of ATP. This process has been referred to as anaerobic glycolysis, but this is truly a misnomer, since the glycolysis occurs in the presence of oxygen which is not utilized in the metabolic pathway. This invention refers to the glycolytic process in RBC as aerobic glycolysis. RBC exclusively utilize this process which is directly related to RBC viability. Viability and drug inhibition of glycolysis can be easily measured by spectrophotometry of free hemoglobin. [5]

At known clinical doses, 2DG monotherapy does not cause RBC hemolysis and therefore the inhibition of aerobic glycolysis is partial, but combined therapy with another potent glycolytic agent is predicted to produce RBC hemolysis.

It is predicted that combined agents that can inhibit aerobic glycolysis can be measured by their effect on RBC viability, which is a very simple and easy measurement compared to cell or tissue culture. This invention proposes that RBC viability can be used to measure the effectiveness of inhibition of aerobic glycolysis, which is a prominent feature of many hypermetabolic disease states. Complete inhibition of aerobic glycolysis may likely cause RBC hemolysis, and therefore may require RBC transfusion.

Aerobic Glycolysis within the Central Nervous System (CNS)

Aerobic glycolysis is intimately linked to L-Lactate production, and this is most significant in the CNS. The astrocyte-neuron L-Lactate shuttle is well described and generally accepted. Elevation of L-Lactate has been shown to be associated with a plethora of mental disorders including panic attacks, PTSD, bipolar disorder, and schizophrenia.[6-10] Some of these conditions can be produced in susceptible individuals by intravenous L-lactate infusion.[11, 12]

Inhibition of aerobic glycolysis in the central nervous system is a special situation because the drugs need to cross the blood brain barrier (BBB). While small molecules such as 2DG cross, larger molecules may need to be converted to an ester prodrug that can transit the BBB and be hydrolyzed by esterases in the CNS. The products of hydrolysis should be the active drug, and a drug familiar to the CNS which effects are known and not deleterious to CNS activity.

### BENEFITS TO SOCIETY

Hypermetabolic states categorize a plethora of serious illnesses. These conditions rely on aerobic glycolysis for ATP production and without sufficient ATP these conditions cannot exist. At the present time, we now have small molecule inhibitors of aerobic glycolysis with acceptable side effects that can be cheaply and easily synthesized. The effectiveness of aerobic glycolysis inhibition can be easily and cheaply monitored in nearly all worldwide health care facilities by spectrophotometric measurements of RBC lysis.

### REFERENCES

1. TeSlaa, T. and M. A. Teitell, Techniques to monitor glycolysis. Methods Enzymol, 2014. 542: p. 91-114.
2. Ganapathy-Kanniappan, S., R. Kunjithapatham, and J. F. Geschwind, Anticancer efficacy of the metabolic blocker 3-bromopyruvate: specific molecular targeting. Anticancer Res, 2013. 33(1): p. 13-20.
3. Katayama, Y., et al., Dichloroacetate, a pyruvate dehydrogenase kinase inhibitor, ameliorates type 2 diabetes via reduced gluconeogenesis. Heliyon, 2022. 8(2): p. e08889.
4. Dikshit, A., et al., Potential Utility of Synthetic D-Lactate Polymers in Skin Cancer. JID Innov, 2021. 1(3): p. 100043.
5. Kahn, S. E., B. F. Watkins, and E. W. Bermes, Jr., An evaluation of a spectrophotometric scanning technique for measurement of plasma hemoglobin. Ann Clin Lab Sci, 1981. 11(2): p. 126-31.
6. Dogan, A. E., et al., Brain lactate and pH in schizophrenia and bipolar disorder: a systematic review of findings from magnetic resonance studies. Neuropsychopharmacology, 2018. 43(8): p. 1681-1690.
7. Maddock, R. J., et al., Elevated brain lactate responses to neural activation in panic disorder: a dynamic 1H-MRS study. Mol Psychiatry, 2009. 14(5): p. 537-45.
8. Kuang, H., et al., Lactate in bipolar disorder: A systematic review and meta-analysis. Psychiatry Clin Neurosci, 2018. 72(8): p. 546-555.
9. Machado-Vieira, R., et al., Increased Brain Lactate During Depressive Episodes and Reversal Effects by Lithium Monotherapy in Drug-Naive Bipolar Disorder: A 3-T 1H-MRS Study. J Clin Psychopharmacol, 2017. 37(1): p. 40-45.
10. Regenold, W. T., et al., Elevated cerebrospinal fluid lactate concentrations in patients with bipolar disorder and schizophrenia: implications for the mitochondrial dysfunction hypothesis. Biol Psychiatry, 2009. 65(6): p. 489-94.
11. Binkley, K. E. and S. Kutcher, Panic response to sodium lactate infusion in patients with multiple chemical sensitivity syndrome. J Allergy Clin Immunol, 1997. 99(4): p. 570-4.
12. Levenstein, S., Lactate infusion induces panic attacks in patients with premenstrual syndrome. Psychosom Med, 1993. 55(1): p. 86-7.

Having described my invention, I claim:

1. A method to determine a drug efficacy of an inhibitor of an aerobic glycolytic process comprised of a measurement of a free hemoglobin molecule in a venipuncture red blood cell sample.

2. The method of claim 1 where the measurement utilizes a spectrophotometer.
